(19) United States
(12) Patent Application Publication
(10) Pub. No.: US 2022/0267161 A1
(43) Pub. Date: Aug. 25, 2022

(54) A Dextran Coated Silica Aerogel Used as a Drug Carrier System and a Dextran Coated Silica Aerogel Production Method

(71) Applicants: Yildiz Teknik Universitesi, Isanbul (TR); Yildiz Teknoloji Transfer Ofisi Anonim Sirketi, Esenler/Istanbul (TR)

(72) Inventors: Sevil YUCEL, Istanbul (TR); Burcu KARAKUZU IKIZLER, Istanbul (TR); Ecem TIRYAKI, Istanbul (TR); Yeliz ELALMIS, Istanbul (TR)

(21) Appl. No.: 17/631,540

(22) PCT Filed: Jul. 22, 2020
(86) PCT No.: PCT/TR2020/050644

§ 371 Date: Jan. 31, 2022

(30) Foreign Application Priority Data
Jul. 31, 2019 (TR) 2019/11602

(51) Int. Cl. C01B 33/154; A61K 47/02; A61K 47/40; A61K 9/12; C01B 33/158

(52) U.S. Cl. CPC C01B 33/154; A61K 47/02; A61K 47/40; A61K 9/122; C01B 33/1585

(57) ABSTRACT

The present invention relates to a dextran (D) coated silica aerogel used as a drug carrier system in colon cancer treatment and a dextran (D) coated silica aerogel production method in which silica aerogels were synthesized with the Sol-Gel method are modified with amine groups and are coated with dextran (D) or dextran aldehyde (DA) to obtain said dextran (D) coated silica aerogel (S).

### TECHNICAL FIELD OF THE INVENTION

The present invention relates to a dextran (D) coated silica aerogel used as a drug carrier system in colon cancer treatment and a dextran (D) coated silica aerogel production method in which silica aerogels were synthesized with the Sol-Gel method are modified with amine groups and are coated with dextran (D) or dextran aldehyde (DA) to obtain the abovementioned dextran (D) coated silica aerogel.

### PRIOR ART

Colon cancer occurs with the normal epithelium in the colon and rectum transforming into an adenomatous polyp. Colon cancer is a type of cancer ranked nr 4 regarding its prevalence in the world. ([1] Aydin, R. and Pulat, M. 2012. 5-Fluorouracil Encapsulated Chitosan Nanoparticles for pH-stimulated Drug Carrier: Evaluation of Controlled Release Kinetics. Journal of Nanomaterials, 42). Every year 1.36 million individuals are diagnosed with colon cancer and nearly one-third of these individuals lose their lives due to colon cancer-related symptoms. ([2] Banerjee, A., Pathak, S., Subramanium, V. D., Dharanivasan, G., Murugesan, R. and Verma, R. S. 2017. Strategies for targeted drug carriers in the treatment of colon cancer: current trends and future perspectives. Drug Discovery Today, 22(8), 1224-1232 [3] Siegel, R. L., Miller, K. D., Fedewa, S. A., Ahnen, D. J., Meester, R. G., Barzi, A. and Jemal, A. 2017. Colorectal cancer statistics, 2017. CA: a cancer journal for clinicians, 67(3), 177-193). According to the report of the WHO—World Health Organization, it is estimated that colon cancer cases will increase by 50% and reach approximately 15 million new cases by 2020. ([4] World Health Organization, 2013).

Many different methods are used in the treatment of colon cancer such as surgery, chemotherapy, and radiation therapy. However, these methods often do not provide enough effectiveness for the recovery of the patient. ([7] Feng, C., Sun, G., Wang, Z., Cheng, X., Park, H., Cha, D., Kong, M. and Chen, X. 2014. Transport Mechanism of Doxorubicin Loaded Chitosan-based Nanogels across Intestinal Epithelium. European Journal of Pharmaceutics and Biopharmaceutics, 87: 197-207, [8] Bose, A., Elyagoby, A. ve Wong, T. 2014. Oral 5-Fluorouracil Colon-Specific Carrier Through In vivo Pellet Coating for Colon Cancer and Aberrant Crypt Foci Treatment. International Journal of Pharmaceutics, 468: 178-186., [9] Liu, K., Wang, Z. Q., Wang, S. J, Liu, P., Qin, Y. H., Ma, Y., Li, X. C. and Huo, Z. J. 2015. Hyaluronic Acid-Tagged Silica Nanoparticles in Colon Cancer Therapy: Therapeutic Efficacy Evaluation. International Journal of Nanomedicine, 10: 6445). Chemotherapy is one of the most important therapeutic methods and different drugs or drug combinations are used to reduce the division of cancer cells during this treatment. However, those chemotherapy drugs being used have many disadvantages such as possible side effects, non-regulation of their accurate dosages, peptide-protein based drugs being possibly affected by the body environment conditions and therefore their limited oral usage, their possible damage to healthy cells and tissues and their improper release, losing their effects before reaching the target area with the desired dosage, being cleared of the blood circulation quickly and their short half-lives. These disadvantages generate the need to develop drug carrier systems which will provide comfortable and effective drug usage to patients during their treatment. Targeting to the colon as a solution to the abovementioned problems can be implemented with pH, time, or enzyme-controlled systems. However, using pH-controlled systems is not enough on its own regarding targeting as the pH levels of the colon and small intestine are very close to each other. Their different periods of transition through the gastrointestinal system causes time-controlled systems to remain limited. For this reason, the development of enzyme-controlled systems functions as an ideal drug delivery system for colon-specific targeting. ([10] Gümü [symbol] derelio\check{g}lu, M. 2004. Dextran-Based Colon Indigenous Drug Release System; Studying Biodegradation and pH Sensitivity's effects on BSA and IgG Release Kinetics. The Scientific and Technical Research Council of Turkey, Ankara., [11] Simonsen, L., Hovgaard, L., Mortensen, P. B. and Brondsted, H. 1995. Dextran Hydrogels for Colon-Specific Drug Carrier, V. Degradation in Human Intestinal Incubation Models. European Journal of Pharmaceutical Sciences, 3: 329-337, [12] [symbol] engel-Türk, C. T., Hasçiçek, C. and Gönül, N. 2006. DRUG CARRIER SYSTEMS TARGETED AT THE COLON. Journal of the Faculty of Pharmacy Ankara, 35(2): 125-148, 2006., [13] Sinha, V. and Kumria, R. 2001. Polysaccharides in Colon-Specific Drug Carrier. International Journal of Pharmaceutics, 224: 19-38).

The usage of molecules like antibodies and growth factors in the targeting of chemotherapy drugs is presented in the literature. However, most of those molecules are high-cost molecules, which require various biotechnology applications. At the same time, the biggest disadvantages of carrier materials are that the materials used as drug carrier systems can be toxic, have low bioavailability, are not biodegradable, their surfaces cannot be functionalized as desired, and they do not allow drug encapsulation in high amounts.

Although researchers all over the world have made great progress in recent years, desired results have not been achieved completely as there are many methods and risk factors. Additionally, the inadequacy of diagnosing methods and the relatively expensive treatment procedures cause an economical burden for the patients having this disease and their families. For this reason, the need of providing innovative approaches and both cheap and radical treatment procedures for the treatment of colon cancer is dramatically increased.

For targeting to the colon that is used for colon cancer treatment today to be successful, it is necessary that the drug carrier system to be used in colon cancer treatment is designed to be able to carry the drug to the colon and that it is not affected by the pH changes at the upper parts of the gastrointestinal (GI) system for the drug not to lose its effectiveness. Colon targeted systems being currently developed as a solution to this problem are; prodrug designs, pH and enzyme sensitive systems, and time-controlled systems ([1] Anitha, A., Sreeranganathan, M., Chennazhi, K. P., Lakshmanan, V. K. and Jayakumar, R. 2014. In vitro Combinatorial Anticancer Effects of 5-Fluorouracil and Curcumin Loaded N, O-Carboxymethyl Chitosan Nanoparticles toward Colon Cancer and in vivo Pharmacokinetic Studies. European Journal of Pharmaceutics and Biopharmaceutics, 88: 238-251-[2] Feng, C., Li, J., Kong, M., Liu, Y., Cheng, X. J., Li, Y., Park, H. J. and Chen, X. G. 2015. Surface Charge Effect on Mucoadhesion of Chitosan-based Nanogels for Local Anti-colorectal Cancer Drug Carrier. Colloids and Surfaces B: Biointerfaces, 128: 439-447).

The first approach in colon targeted systems is based on oral prodrug application and the prodrug is metabolized to the active drug by the stomach enzyme and microflora while it passes through the small intestine and shows its effect against cancer when it reaches the colon area. The second approach is developing drug carrier systems based on the time period of transition from the upper GI tract to the colon area. Anti-cancer drugs are coated with various materials to form micro-spheres to reduce the dissolution rate and to make them able to stay in an aqueous solution for a long time without dissolving. The third approach is developing a pH-sensitive carrier system using pH-sensitive polymers. In this approach, the drugs are encapsulated with pH-sensitive polymers, which can tolerate acidic environments and can only release the drugs in neutral pH environments. In the fourth approach, systems, which are sensitive to the microflora activated by the colonic enzymes, are used. The microbial enzymes in the colon area enable active ingredients of the drug to be released in the colon environment by disintegrating the polymer substrate. ([3] Gümü [symbol] derelio\check{g}lu, M. 2004. Dextran-Based Colon Indigenous Drug Release System; Studying Biodegradation and pH Sensitivity's effects on BSA and IgG Release Kinetics. The Scientific and Technical Research Council of Turkey, Ankara—[4] [symbol] engel-Türk, C. T., Hasçiçek, C. and Gönül, N. 2006. DRUG CARRIER SYSTEMS TARGETED AT THE COLON. Journal of the Faculty of Pharmacy Ankara, 35(2): 125-148, 2006.—[5] Sinha, V. and Kumria, R. 2001. Polysaccharides in Colon-Specific Drug Carrier. International Journal of Pharmaceutics, 224: 19-38.)

Various drug carrier systems using natural polymers including polysaccharides (alginate, hyaluronic acid, dextran (D) and chitosan), and various proteins (collagen, albumin, elastin, and gelatin) are being developed. Eudragit® polymer, which is developed for reaching this object, is a colon-specific, pH-sensitive polymer. While this polymer does not dissolve in acidic environment pH, by dissolving in pH 6 and above it enables the drug being delivered to be protected from the stomach environment. ([5] Sinha, V., and Kumria, R. 2001. Polysaccharides in Colon-Specific Drug Carrier. International Journal of Pharmaceutics, 224: 19-38.)

However, it is very important for an effective treatment that the delivery systems to be developed today have many features such as effective targeting at the colon area, providing the possibility of loading high amounts of drugs, not showing toxic effects for the body, being biodegradable, having cheap and easy synthesis to apply. The drug carrier systems developed in many studies do not have these features and usually targeting studies are made with high-cost biomarkers, which increases the cost of the treatment. In addition, the drug release rate may be too fast or too slow and the dosage of the active ingredient of the drug cannot be controlled.

### BRIEF DESCRIPTION OF THE INVENTION

The object of the present invention to implement a production method to produce a dextran (D) coated silica aerogel, in which the surfaces of aerogels which are synthesized with the sol-gel method are modified with amine groups and after that are coated with dextran (D) and dextran aldehyde (DA) separately in order to be used as a drug carrier system which is not affected by the stomach and intestine pH, however, is dextranase enzyme controlled and colon targeted.

### DETAILED DESCRIPTION OF THE INVENTION

“A dextran (D) coated silica aerogel used as a drug carrier system and a dextran (D) coated silica aerogel production method” which is conducted to achieve the object of the present invention, are shown in the figures; wherein

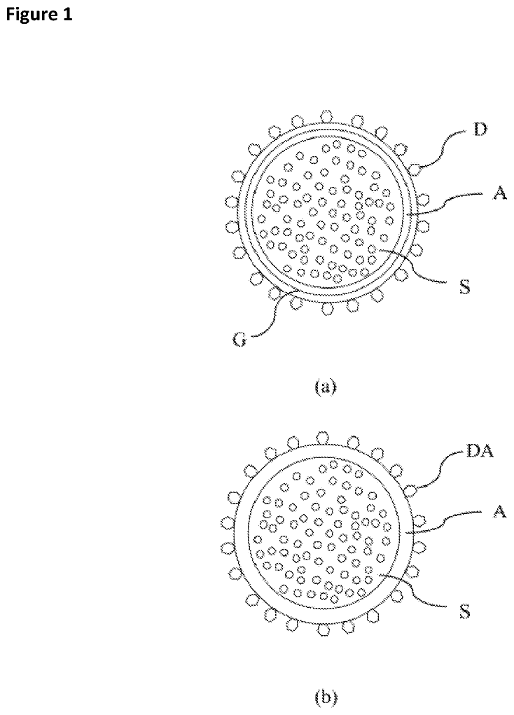

FIG. 1 illustrates the appearance of the dextran (D) coated silica aerogel (a) and dextran aldehyde (DA) coated silica aerogel (b) of the inventive production method.

The production method of a dextran (D) coated silica aerogel used as a drug carrier system comprises the process steps of;
Surface modification of silica aerogels synthesized by sol-gel method,
The modified silica aerogels (S) being coated with dextran (D) or dextran aldehyde (DA).

In the inventive production method, for the dextran-silica conjugation using glutaraldehyde (G) crosslinker, a “Schiff Base” reaction is carried out between the amine groups on the surfaces of the APTES modified silica aerogels (S) and the aldehyde groups of glutaraldehyde, and dextran (D) is being coated on the silica surface by means of glutaraldehyde (G).

Also, for the dextran aldehyde-silica aerogel conjugation, glutaraldehyde (G) crosslinker is not needed and stable imide links are formed between the aldehyde groups of the dextran aldehyde (DA) structure and amine groups of the APTES modified silica aerogel surfaces. With the inventive production method, by means of the dextran (D) and dextran aldehyde (DA) coated on the surface of aerogels; the drug molecules loaded on the aerogels, while not being released during passage through the gastrointestinal system, are only released following the degradation of dextran (D) at the colon area where dextranase enzymes breaking down dextran (D) are found.

In the inventive production method, silica aerogels (S) are synthesized with a sodium silicate solution. In the drying step, an air spray dryer is used, thus, the collapse of the pores in the texture of the hydrophilic silica aerogels is prevented.

In the inventive production method, to obtain silica aerogels originating from sodium silicate, preferably 1:10 proportioned sodium silicate:water is mixed at room temperature and sol is composed. During the mixing procedure, the gelation pH is adjusted by adding 1 M HCl so that the pH value is 4. With the aim to strengthen the network structure, the gel is aged in the air dryer at 50° C. preferably for 24 hours, and after the aging step in order to remove the salts in the gel structure, washing with water is implemented. Preferably the washing process should be repeated 3 times. After washing with water in order to remove the water in the pores, preferably three times washing with ethanol is implemented and gels which are put into ethanol after washing are kept in the air dryer at 50° C. for 24 hours. For 24 hours, the washing process with ethanol is repeated 3 times. At the end of 24 hours, in order to remove the ethanol from the pores of the gel washing process with n-hexane is implemented 3 times and gels are kept in n-hexane in the air dryer at 50° C. for 24 hours and during the 24 hour period, the washing with n-hexane is repeated 3 times. The synthesized silica aerogels are dried in an air spray dryer set to 190° C. inlet temperature and 80° C. outlet temperature.

In the inventive production method, the surface of the synthesized silica aerogels (S) is modified by using APTES. In the invention, for the optimization of the silanization reaction of the silica surface with the APTES, the optimum APTES concentration information is reached by working at different APTES concentrations. For this, 500 mg silica aerogel (S) powder is ultrasonically dispersed in 250 ml toluene and the mixture obtained by adding APTES at different proportions (0.5%, 1%, 2%, 5%, 10%) into the solution, is mixed with a magnetic stirrer preferably 24 hours at 75° C. temperature. At the end of the mixing process which lasts 24 hours, with the mixture reaching room temperature, the unbonded silane agents are removed from the mixture. For this, the mixture is subjected to washing with toluene and preferably centrifuged at 4800 rpm' for 10 minutes and the washing process is repeated preferably three times.

The amine-modified silica aerogels obtained after washing, are dried at 80° C. in an air-drying oven.

Syntheses Stages, (a) Synthesis of silica aerogels, (b) The modification of the surface of the silica aerogel with APTES and Its loading with the drug 5-FU, (c) The coating of silica aerogel surface with dextran (D) and dextran aldehyde (DA).

In order to compare the drug adsorption capacities of the silica aerogels (S) obtained by the production method of the present invention, in the experimental study, drug loading is carried out into the pure silica aerogel (SiO₂) and amine-modified (SiO₂—NH₂) silica aerogels (S), separately. In the experimental study, 5-Fluorouracil (5-FU), which is used in colon cancer treatment, is used as a model drug. Besides this, in order to observe effects of the change of the electrostatic interaction between the amine groups and 5-FU adherent to the pH, to the loading capacity, the loading solution in which the silica aerogels modified with amine groups (S) were, is sonicated for five minutes in the ultrasonic bath with the pH being adjusted to pH 5 and pH 8 and it is enabled that aerogels are dispersed homogeneously within the solution. In the experimental study, the loading process is carried out with magnetic stirrer being 24 hours at room temperature. At the end of 24 hours, the drug-loaded silica aerogels (S) are centrifuged and are left to dry on an air-drying oven at 50° C. for 24 hours.

In order to observe the effect of differently concentrated solutions to the loading behavior, the 5-FU/water solutions are prepared preferably 3 mg/ml and 6 mg/ml, and the effect of the drug concentration on the loading behavior is observed. It is enabled that the moisture on the surface of the silica aerogels is removed by having kept the silica aerogels (S) at 105° C. for 1 hour before drug loading. The loading process is carried out by adding 0.1 gram silica aerogel (S) into 50 ml of drug solutions with different drug concentrations and mixed for 24 hours at room temperature by a magnetic stirrer. At the end of hours, the drug-loaded silica aerogel (S) mixture is centrifuged for 10 minutes and it is enabled that the supernatant keeping the drug molecules which have not been loaded to the silica aerogels are separated. The absorbance of the supernatant is measured with the “Liquid Chromatography-Mass Spectrometer” device and the loaded drug concentration into silica aerogels (S) is calculated. The drug-loaded aerogels which were separated from the supernatant are dried in an air-drying oven at 50° C. for 24 hours.

In the inventive production method, in order to coat the surfaces of silica aerogels (S) with dextran (D), cross-linking with dextran (D)-glutaraldehyde (G) and linking with dextran aldehyde (DA) was used. For the coating procedure, the silica aerogels (S) which are functionalized with amine groups and loaded with the model drug first, are coated with dextran (D) (Mw: 75000) using glutaraldehyde (G) crosslinker. The optimization of the concentration of the glutaraldehyde in which silica aerogels are coated the best is made by preparing glutaraldehyde (G) solutions at different concentrations. A Sodium bicarbonate solution (NaHCO₃) at pH 8.5, which is the appropriate pH for the Schiff Base reaction to occur between the amine groups bonded to silica aerogels (S) and the aldehyde groups in glutaraldehyde (G), is prepared (50 ml) and aerogel particles (0.1 g) are dispersed homogenously within the solution.

Glutaraldehyde (G) at different proportions (5% and 10% w/w GA/solution) is added and the mixture is mixed for 3 hours at room temperature and at the end of the mixing process, it is enabled that the reaction is stopped and stable secondary links are formed by adding cyanoborohydride (NaBH₃CN)to the mixture. The particles obtained at the end of the reaction are dialyzed against distilled water overnight using the dialysis membrane and the silica aerogels (S) the surfaces of which were linked with glutaraldehyde (G) are dried on the air drying-oven at 50° C. Dextran (D) is dissolved in potassium chloride (KCl) solution (50 ml) with a 2 pH. Glutaraldehyde (G) linked silica aerogels (S) (0.1 g) are added into the dextran (D) solution and mixed for 24 hours at room temperature and the dextran (D) coated silica aerogels obtained at the end of the reaction are washed with water in order to remove the non-linked molecules and are subjected to the drying process on an air drying-oven at 50° C.

And in the procedure of linking with dextran aldehyde (DA), for the dextran aldehyde (DA) synthesis, 3.3 gram dextran (D) (Mw: 75000) is dissolved in 30 ml distilled water. 8 grams of sodium (meta) periodate (NaIO₄) is dissolved in 70 ml distilled water. The NaIO₄ solution has slowly been added to the dextran (D) solution and is mixed with a magnetic stirrer for 24 hours in the dark at room temperature. At the end of 24 hours, in order to remove the excess NaIO₄, washing against distilled water overnight using the dialysis membrane in the dark at room temperature is applied. The solution obtained after the washing is dried by freezing with a freeze dryer. The dextran aldehyde (DA) synthesized in the former step so that the silica aerogels (S) are coated, is dissolved in distilled water (50 ml). The drug-loaded silica aerogels (0.1 g) are put into the dextran aldehyde (DA) solution, the pH of the solution is adjusted to 8.5 and it is mixed for 24 hours at room temperature with a magnetic stirrer. At the end of 24 hours, cyanoborohydride (NaBH₄) (1 mg/ml) is added into the mixture and is mixed for 15 minutes. At the end of 15 minutes again NaBH₄ (1 mg/ml) is added and it is enabled that the mixture is mixed for 30 more minutes. In order to remove substances that did not go into reaction, washing with distilled water using a dialysis membrane is carried out overnight. The dextran aldehyde (DA) coated silica aerogels (S) obtained after washing, are dried on an air drying-oven at 50° C.

In the experimental study, the dialysis membrane method is used for the study of the in vitro release of the 5-Fluorouracil drug from the silica aerogels (S). First, for the studies of release in the enzyme-free environment, silica aerogels (S), which are dextran-coated and not coated, are put in simulated gastric fluid, the release behavior in gastric pH (1.2) and the effect of dextran (D) coating on the release are analyzed. At the end of two hours, the aerogels were put in simulated intestinal fluid, the pH of which was 6.8, and were held there for 15-18 hours and the release behaviors of the drug-loaded silica aerogels (S) against the changes of pH in the gastrointestinal system were compared.

Secondly, for the studies of release in an environment with an enzyme, in order to study the release of dextran (D) coated silica aerogels (S) depending on the presence of the enzyme, the release profile of silica aerogels (S) in a release environment containing dextranase (acetate buffer, pH 5.5) is studied. In order to define the concentration of 5-FU released from the silica aerogels (S) cumulative release values were calculated by time-dependent measurements taken with a “Liquid Chromatography-Mass Spectrometer” device.

As a result of experimental studies, silica aerogels (S) having a high surface area (520.53 m²/g) and pore diameters (5.73 nm) have been synthesized and it is observed that its surface being modified with amine has increased the drug loading and dextran (D) coating efficiency. It is seen that high drug loading efficiency occurred at pH 8 and when APTES (A) at the proportion 10% was used (611.96 mg/g drug/silica aerogel). The model drug 5-FU release behaviors of silica aerogels (S) coated in two different ways with dextran-glutaraldehyde and dextran aldehyde (DA), in the stomach (pH=1.2), intestines (pH=6.8) and colon tumor (pH=5.5) environments are studied separately. As a result, pure silica aerogels (S), which were not coated with dextran (D), have released 85-86% of the 5-FU drug at stomach-intestine and colon pHs within 24 hours. The release of the drug 5-FU from silica aerogels (S) after being coated with dextran (D) and dextran aldehyde (DA) in the stomach and intestine fluids within 24 hours has occurred at the values 1.68% and 3.49% respectively while occurring at enzyme-free colon pH 4.21% and 0.97% respectively.

The effect of the presence of enzyme to the release is studied by adding 2 U/ml dextranase enzyme in the acetate buffer which provided colon pH. 24 hours after the enzyme is added the release of drug 5-FU from dextran (D) and dextran aldehyde coated silica aerogels has shown an increase being 24.02% and 13.42% respectively. The coating of silica aerogels (S) with dextran (D) which is a natural polymer and dextran aldehyde (DA) which is a dextran derivative enables the 5-FU drug to reach the colon area without being affected by the pH changes and losing its effect and a controlled release with the effect of enzyme.

The silica aerogels (S) (SiO₂) used in the inventive production method, are used as ideal drug carrier systems due to their high surface area and large pore volumes and their biocompatible structures having high porosity and their high adsorption capacity.

Due to the surfaces of silica aerogels (S) (SiO₂) being able to be modified easily, by coating their surfaces with dextran (D) a controlled and colon targeted, potential drug carrier system is obtained.

Dextran (D), which is a biodegradable and biocompatible natural polymer is a polysaccharide. It is being preferred because of its features such as its wide molecular weight distribution, not being toxic, and being able to be eliminated from the body easily.

In the inventive production method, the surface of the silica aerogels (S) is modified with preferably 3-(aminopropyltriethoxysilane) (APTES) (A) (SiO₂—NH₂). Amine groups give the Schiff Base reaction with the aldehyde groups of glutaraldehyde, which is used as a crosslinker for dextran (D) coating (Silica Aerogel (S) @3-Aminopropyltriethoxysilane (APTES) (A)-SiO₂—NH₂). For the dextran (D) coating of the surface of silica aerogel (S), glutaraldehyde (G) crosslinker is used and the capacity of glutaraldehyde (G) bonding to the surface affects the dextran (D) coating efficiency directly. The coating of the surfaces of silica aerogels (S) with dextran (D) occurs between the aldehyde groups of the glutaraldehyde (G) bonded to the surface and hydroxyl groups of dextran (D) at acidic conditions. (Silica Aerogel (S) @ APTES (A) @ Glutaraldehyde (G)-(SiO₂—NH₂-GA))

With the inventive production method, due to the surfaces of the silica aerogels (S) being coated with dextran (D), the release of the drug-loaded to the aerogels in the stomach and intestine fluids is prevented and colon targeted release is made. Thus, the dextran (D) coated onto the surface of the silica aerogels (S) being decomposed by only the dextranase enzymes present in the colon, the targeted release of the drug is enabled. (Silica Aerogel (S) @ APTES (A) @ Glutaraldehyde (G) @ Dextran (D)-(SiO₂—NH₂-Dex))

In the inventive production method, the surfaces of silica aerogels (S) are also coated with dextran aldehyde (DA) without using glutaraldehyde (G) crosslinker. Dextran aldehyde (DA) links to the amine groups on the surface of silica aerogel, with the aldehyde groups it has, with a Schiff Base reaction and enables the silica aerogel (S) carrying the drug to the colon effectively. (Silica Aerogel (S) @ APTES (A) @ Glutaraldehyde (G) @ Dextran Aldehyde (DA)-(SiO₂—NH₂-Dex-CHO))

With the inventive production method, dextran (D) coated silica aerogel (S) used in colon cancer treatment is obtained. Dextran (D) coated silica aerogel (S) is being used as a drug system for colon cancer treatment. In the production of dextran (D) coated silica aerogel (S), silica aerogels with high adsorbing capacity are used in order to be used in drug carrier system and silica aerogels are synthesized with sodium silicate which is biocompatible and economical, using the sol-gel method instead of organic structured, costly, toxic and expensive silica sources such as Tetraethylorthosilicate (TEOS), Tetramethylorthosilicate (TMOS) which are used during the synthesizing of many carrier systems and in the synthesizing of silica nanoparticles.

By this means, opposite to many studies present in the previous technique, drug loading at rather high amounts is implemented.

Although colon targeted carrier systems with dextran hydrogels, drugs conjugated with dextrans (D), polysaccharide-based carriers like pectin and chitosan, silica and similar nanoparticles and drug conjugates are present in the literature, a production method where silica aerogels are used with the aim of carrying to the colon and silica aerogels (S) are conjugated with dextran (D) is not present in the previous technique. With the inventive production method dextran (D)-silica aerogel (S) and dextran aldehyde (DA)-silica aerogel (S) organic-inorganic hybrid systems, which are new and effective, are obtained.

In the inventive production method, the process of coating dextran (D) and dextran aldehyde (DA), which is a derivative of dextran (D) onto silica aerogels (S), eliminates the necessity of using high-cost material as it takes place in the previous technique. Although the usage of molecules such as antibody, growth factor in the targeting of the chemotherapy drugs is present in the literature, the process of coating silica aerogels (S) with dextran (D) and dextran aldehyde (DA), a derivative of dextran (D), which are not affected by the acid environment and is inhibited by dextranase enzymes indigenous to the colon, reduces the cost and it is ensured that the drug is stably targeted to the colon.

The dextran (D) coated silica aerogel (S) obtained with the inventive production method and used as a drug carrier system can be taken orally, contrarily to the chemotherapy drugs which are applied via injection, used today, thus it is prevented that the drugs are removed from the body in a short time. This way, it is ensured that they reach the tumor area in the colon, and with the reduction of side effects based on chemotherapy drugs, healthy cells are not damaged and a more effective treatment application at one time is enabled. By means of the dextran (D) coated aerogels obtained with the inventive production method, a prolonged drug release is practiced, and the release rate can be controlled by adjusting the thickness of the layer of dextran (D) coated onto the surface.

It is possible to develop various versions of the dextran (D) coated silica aerogel to be used as a drug carrier system and a dextran (D) coated silica aerogel production method (1) which are the subjects of the invention, however, the invention cannot be limited by the examples explained herein and is as defined in the claims.

1. A production method of a dextran (D) coated silica aerogel that is used as a drug carrier system, the method comprising the steps of:
modifying surfaces of silica aerogels (S) which are synthesized by sol-gel method, and
coating the modified silica aerogels (S) with dextran (D) or dextran aldehyde.

2. A dextran-coated silica aerogel production method according to claim 1, wherein a Schiff Base reaction is carried out between amine groups on the surface of the silica aerogels (S) modified with 3-(aminopropyl)triethoxysilane (A) by using glutaraldehyde (G) crosslinker for the dextran (D)-silica aerogel (S) conjugation, and aldehyde groups in the glutaraldehyde and dextran (D) is coated to silica surface by means of glutaraldehyde (G).

3. A dextran-coated silica aerogel production method according to claim 1, wherein stable imide bonds are created between the aldehyde groups of the dextran aldehyde (DA) structure and the amine groups of the APTES modified silica aerogel surface, for the dextran aldehyde (DA)-silica aerogel (S) conjugation.

4. A dextran (D) coated silica aerogel production method according to claim 1, wherein sodium silicate and water mixing preferably at a ratio of 1:10 at ambient temperature and creating sol to obtain sodium silicate sourced silica aerogels (S).

5. A dextran-coated silica aerogel production method according to claim 4, wherein gelation pH is adjusted by adding 1 M HCl so that pH value is 4 during the mixing procedure.

6. A dextran-coated silica aerogel production method according to claim 4, wherein the gel is aged in an air dryer at 50° C. preferably for 24 hours with the aim to strengthen the network structure of the gel obtained and washed with water in order to remove the salts within the gel structure after the aging step.

7. A dextran-coated silica aerogel production method according to claim 6, wherein a washing process is carried out with ethanol for preferably three times in order to remove the water in the pores after washing with water and gels which were put into ethanol, are kept in an air dryer at 50° C. for preferably 24 hours.

8. A dextran-coated silica aerogel production method according to claim 7, wherein a washing operation is performed with n-hexane three times in order to remove the ethanol in the pores of the gel and the gels are kept in an air dryer at 50° C. in n-hexane and repeating the washing with n-hexane for three times within 24 hours.

9. A dextran-coated silica aerogel production method according to claim 8, wherein the synthesized silica aerogels (S) are dried in an air spray dryer, the inlet temperature of which is adjusted to 190° C. and outlet temperature is adjusted to 80° C.

10. A dextran-coated silica aerogel, used as a drug carrier system in colon cancer treatment.

11. A dextran aldehyde coated silica aerogel, used as a drug carrier system in colon cancer treatment.
